(19) United States
(12) Patent Application Publication
(10) Pub. No.: US 2004/0086904 A1
(43) Pub. Date: May 6, 2004

(54) Screening assay for cotranslational translocation interfering compounds

(76) Inventors: Christiane Dascher-Nadel, Marseille (FR); Ivan James Dalton Lindley, Brunn/Gebirge (AT); Kathi Franziska Marquardt-Gehrke, Braunschweig (DE)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/466,725

(22) PCT Filed: Jan. 21, 2002
(86) PCT No.: PCT/EP02/00569

(30) Foreign Application Priority Data
Jan. 22, 2001 (GB) 0101598.1

(51) Int. Cl. C12Q 1/68

(52) U.S. Cl. 435/6

(57) ABSTRACT

An assay comprising cells containing DNA which encodes a fusion protein containing a signal peptide fused and/or linked to a reporter gene protein for the identification of compounds which interfere with the process of cotranslational translocation and with the production of secreted or membrane protein.

The present invention relates to a screening method, i.e. a process for the screening of compounds, e.g. organic compounds, such as to a process for the identification of compounds which interfere in the production of secreted and/or membrane proteins in a cell, e.g. which inhibit the production of secreted and/or membrane proteins in a cell. Such compounds may be useful as pharmaceuticals, e.g. in the treatment of diseases which are based on the (non)production of a secreted or membrane protein in a cell, e.g. diseases which are mediated by a secreted or membrane protein in a cell.

Processes for the identification of compounds which interfere in the production of secreted and/or membrane protein have been proposed already. Such processes may be rather substrate specific and in general may not be generalised. We have now found a novel process for the identification of compounds which interfere in the production of secreted and/or membrane protein by control of cellular protein production in the process of cotranslational translocation across the membrane of the endoplasmatic reticulum (ER). We thus have surprisingly identified a new level of control of cellular protein production which may be used in a process for the identification of compounds which interfere with, e.g. inhibit, synthesis of secreted and/or membrane proteins in cells. A process according to the present invention is not substrate specific and is universally applicable.

Cotranslational translocation across and insertion into the membrane of the ER is one of the early steps in translational synthesis of most secreted and membrane proteins, e.g. in eukaryotic cells (Matlack et al., 1998). Nascent secretory and membrane proteins destined for export from the cell through the classical ER/Golgi pathway are recognised and targeted to the ER membrane when a hydrophobic leader sequence or signal peptide (SP) emerges form the ribosome. The signal recognition particle (SRP) binds to the SP and ribosome, thereby temporarily halting continued protein chain elongation. Targeting of the ribosome-nascent chain/signal sequence-SRP complex to the membrane of the ER through the interaction of SRP with its specific receptor (SR) releases translational arrest. The ribosome-nascent chain complex is transferred to the translocation channel (translocon, Sec61 complex) a highly complex structure formed by several membrane proteins with different functions and polypeptide chain elongation resumes. Subsequent insertion of the nascent chain into the translocation channel results in formation of a stable complex between the ribosome and the translocon. The nascent chain is then translocated through the aqueous translocon channel, the SP is integrated into the ER membrane and in most cases cleaved off. Concomitantly with translocation N-linked glycosylated proteins are core-glycosylated in the lumen of the ER and existing transmembrane domains are inserted into the ER membrane.

It was now found that the production of secreted and membrane proteins is inhibited by specific interference of inhibitor compounds with the process of cotranslational translocation of the nascent secreted or membrane protein chain and that the SP plays the crucial role in this process. Thus, it was surprisingly found that the target protein of this compound class may be a component of the translocation machinery localised in the ER membrane, which recognises and interacts with the SP of the secreted or membrane protein, thereby assisting the nascent protein chain to cross the membrane. The finding of cotranslational translocation as a new level of regulation of protein expression suggests that other structural subclasses of SPs may exist in other proteins, and may interact with the translocation machinery in a different, but specific manner. Therefore, this interaction may be utilised to identify substances specifically interfering with this event and the subsequent production of the respective protein. We have further found that the same finding as described above is obtained when all or part of the non-SP portion of a protein, i.e. the amino acid sequence between the C-terminal amino acid of the SP and the C-terminal amino acid of the secreted or membrane protein, is replaced by a reporter protein e.g. a protein which may be detected by use of detection methods available. This supports the notion that the cotranslational translocation process is essentially independent of the nature of the secreted or membrane protein, but is dependent upon the nature of the SP. We have further found that, when DNA encoding such SP-reporter gene fusions is introduced into living cells and expressed using known methods e.g. by introducing the DNA to the 3′-end of a mammalian promoter in a mammalian expression vector, in the absence or in the presence of a candidate compound, the secretion of the reporter protein into the extracellular environment mirrors the candidate compound sensitivity of that protein from which the signal peptide is derived. DNA constructs encoding a reporter protein fused to, and therefore directed to, the translocation machinery of a cell by a selected heterologous SP can thus be used for the screening of candidate compounds to obtain compounds which interfere with the production of that protein from which the signal peptide is derived.

In one aspect the present invention provides the use of a DNA construct encoding a reporter protein fused to a selected heterologous signal peptide, e.g. of a secreted and/or membrane protein; e.g. fused to and directed to the translocation machinery of a cell by a selected heterologous signal peptide; in a screening process or screening assay for the screening of candidate compounds, to obtain compounds which interfere with the process of cotranslational translocation and with the production of, e.g. said, secreted and/or membrane protein, e.g. compounds which interfere with the production of that protein from which the selected signal peptide is derived, e.g. pharmaceutically useful compounds.

DNA encoding said reporter protein fused to a selected heterologous signal peptide may be cloned into an appropriate expression vector and the vector obtained may be introduced into living cells. Cells thus obtained may be used for screening purposes, resulting in an assay system which enables the identification of compounds which are pharmaceutically useful, e.g. in the therapy/prevention of a disease mediated by a secreted or membrane protein exported via the classical ER/Golgi pathway. According to the present invention compounds which interfere with the function of the translocation machinery and interfere specifically with the process of cotranslational translocation of secreted and/or membrane proteins carrying SPs may thus be identified by quick and simple means.

In another aspect the present invention provides a process for the identification, e.g. and selection, of a compound which interferes with the production of secreted and/or membrane protein comprising determining the amount of protein secreted and the extent or degree of cotranslational translocation across the membrane of the endoplasmatic reticulum of said secreted and/or membrane protein in the presence and in the absence of a candidate compound.

Means for such process may be included in an assay or a kit.

In another aspect the present invention provides a process for the identification, e.g. and selection, of a compound which interferes with the process of cotranslational translocation and with the production of secreted and/or membrane protein by a cell, comprising the steps of

a. providing DNA which encodes a fusion protein containing, e.g. consisting of, a heterologous signal peptide linked to a reporter protein, e.g. with or without additional intervening DNA sequences which encode additional amino acids; such as additional sequences from the adjacent mature sequence,

b. introducing DNA obtained in step a. into a DNA vector, e.g. a mammalian expression vector;

c. transfecting DNA obtained in step a. or in step b. into a cell;

d. allowing or stimulating expression of the reporter gene protein in a transfected cell obtained in step c. under appropriate conditions;

e. detecting secreted reporter protein produced in step d. in the absence or in the presence of a candidate compound, respectively; and

f. determining whether there is a difference in the amount of reporter gene protein produced in step e. in the absence or in the presence of a candidate compound, respectively, which amount is determined according to step e.; e.g. and optionally

g. selecting a candidate compound, in the presence of which the amount of reporter gene protein produced in step e. is different from the amount of reporter gene protein produced in step e. in the absence of said candidate compound, e.g. and using such selected compound as a pharmaceutical.

In another aspect the present invention provides a process for the identification, e.g. and selection, of a compound which interferes with the process of cotranslational translocation and with the production of secreted and/or membrane protein by a cell comprising the steps of

a1. allowing or stimulating expression of the reporter gene protein in cells transfected with DNA which encodes a fusion protein containing, e.g. consisting of, a heterologous signal peptide linked to a reporter protein, e.g. with or without additional intervening DNA sequences which encode additional amino acids under appropriate conditions; such as additional sequences from the adjacent mature sequence,

b1. detecting secreted reporter protein produced in step a1. in the absence or in the presence of a candidate compound, respectively, and determining whether there is a difference in the amount of reporter gene protein produced in step a1. in the absence or in the presence of a candidate compound, respectively, e.g. and optionally

c1. selecting a candidate compound, in the presence of which the amount of reporter gene protein produced in step a1. is different from the amount of reporter gene protein produced in step a1. in the absence of said candidate compound, e.g. and using such selected compound as a pharmaceutical, e.g. after chemical derivatisation.

If a difference is determined in step f., or b1, respectively, the candidate compound evidently interferes in the process of cotranslational translocation and thus, in the production of secreted and/or membrane protein in a cell. A candidate compound, in the presence of which the amount of reporter gene protein produced in step e., or step a1, respectively, is different from the amount of reporter gene protein produced in the absence of said candidate compound may be useful as a pharmaceutical, e.g. in the therapy/prevention of a disease mediated by a secreted or membrane protein exported via the classical ER/Golgi pathway. Candidate compounds which may interfere in the production of secreted or membrane protein, e.g. in cotranslational translocation, include e.g. libraries of chemicals and natural extracts, low molecular weight (LMW) compounds, peptides, antibodies, recombinant DNA molecules and expression libraries, DNA, RNA etc. A “signal peptide” as used herein includes a peptide/protein sequence that is able to export a secreted and/or membrane protein via the ER/golgi pathway, e.g. a signal peptide as such, a signal peptide comprising signal anchors, etc.

Generally, DNA molecules encoding proteins may be obtained as appropriate, e.g. by a method as conventional, e.g. by cloning from a cDNA or genomic DNA library, by polymerase chain reaction (PCR) amplification and cloning, e.g. obtained from commercial sources or from the ATCC/NIH repository of human DNA probes. Nucleotide sequences of proteins are generally available from public databases such as Genbank and EMBL or publications.

An appropriate reporter protein includes a protein that

allows convenient and sensitive detection of said protein e.g. in cell culture media containing said protein, and

does not interfere with the process of cotranslational translocation of said fusion protein and subsequent export thereof,

e.g. placental secreted alkaline phosphatase (SEAP).

A fusion protein containing a heterologous signal peptide linked to a reporter protein, e.g. with or without additional intervening DNA sequences which encode additional amino acids, hereinafter designated as “a fusion protein according to the present invention”, may be prepared as appropriate, e.g. according to the POR-ligation-PCR mutagenesis method (Ali and Steinkasserer, 1995). Methods for subcloning into an appropriate vector expression system may be carried out as appropriate, e.g. according, e.g. analogously, to a method as conventional, e.g. including standard procedures.

Additional intervening DNA sequences which encode additional amino acids under appropriate conditions may include e.g. parts of the DNA sequence from the mature DNA sequence adjacent to that signal peptide of the secreted and/or membrane protein which is part of the fusion protein used in a process of the present invention according to step a. or step a1., respectively.

An appropriate vector system may comprise

an efficient promoter element for transcription initiation, either constitutive or inducible,

a transcription terminator,

a polyadenylation (poly(A)) signal sequence,

bacterial origin of replication,

selectable markers for bacterial propagation and for selection of mammalian cells that have stably integrated the plasmid DNA.

Appropriate efficient promoter elements for transcription initiation, either constitutive or inducible, transcription terminators, polyadenylation (poly(A)) signal sequences, and bacterial origin of replication may be dependent on the nature of the host cell used and may be chosen as appropriate, e.g. according, e.g. analogously, to a method as conventional. An appropriate selectable marker includes a gene that confers a phenotype on the host cell that allows transformed cells to be identified and preferably allows a growth advantage under specified conditions. Appropriate selectable markers for bacteria are well known and e.g. include resistance genes for ampicillin, kanamycin, and tetracycline. For the establishment of stable mammalian cell lines appropriate selectable markers may be used, e.g. well known selectable markers, e.g. including hygromycin, neomycin. An appropriate expression vector which may be transfected into host cells may be chosen as appropriate, and may be transfected into host cells as appropriate, e.g. according, e.g. analogously, to a method as conventional, or by a method as described herein. An appropriate host cell includes a host cell that is compatible with the vector and proficient to drive expression of the recombinant cDNA fusion genes from either the selected constitutive or inducible promoter. Expression of the fusion protein according to the present invention may be e.g. either transient or after stable integration into the host genome. Transient expression is a convenient and rapid method to study expression of recombinant genes in mammalian cells. In general, when cells acquire DNA, they express it transiently over a period of several days to several weeks before the DNA is eventually lost from the population. Selection for stable integration of plasmid DNA into the host chromosome permits the generation of stably transfected cell lines that indefinitely express a desired recombinant gene product. Transient transfection protocols and protocols for generation of stable cell lines are known, e.g. and include electroporation and transfection, e.g. mediated by commercially available transfection reagents such as cationic phospholipids (e.g. Lipofectamin®, Boehringer), activated dendrimers (Superfect®, Qiagen) etc. Recombinant SP-reporter gene cDNAs can be expressed either constitutively or inducibly. The advantages of constitutive promoter elements such as e.g. the cytomegalovirus (CMV) immediate-early or late promoter are that they are very active in a wide variety of cell types and ensure high levels of expression without any additional external stimuli. Inducible systems that permit controlled induction of gene expression on the other hand ensure expression of the recombinant cDNA only when desired. Preferred promoters express the fusion protein according to the present invention at high levels. Test cells expressing the fusion protein according to the present invention preferably also express a cytosolic specificity/toxicity control protein, e.g. luciferase, from a promoter which is the same or which has the same specific function (i.e. initiating transcription), as the promoter of the fusion protein according to the present invention. When treated appropriately such cells will produce the cytosolic specificity/toxicity control protein which is not secreted (no export). Such cells may be obtained as appropriate, e.g. according, e.g. analogously, to a method as conventional. Appropriate assays for detection of secreted and cytoplasmic reporter proteins in cell-based assays may be used, e.g. including Western Blot, ELISA and colorimetric or fluorescence-based methods for detecting enzymatic reporter proteins such as secreted placental alkaline phosphatase or luciferase. In all such assays the test cells expressing the fusion protein according to the present invention are incubated with and without a candidate compound, respectively.

Once a test cell has been constructed, an inhibitor of export, i.e. a candidate compound which inhibits export (=inhibitor), may be identified by an appropriate cell-based screening assay, e.g. including assays as described herein. In a preferred assay a cell expressing the fusion protein according to the present invention is treated with a candidate compound and the amount of secreted reporter protein is compared to the amount determined without treatment. A compound is regarded to inhibit export (=inhibitor) if there is a reduction in the amount of protein detected extracellularly, at the cell surface or in the cell supernatant, in the assay performed in the presence of the inhibitor compared to the assay performed without the inhibitor. Preferably, the inhibitor reduces export of the reporter protein by at least 50%, even more preferably 80% or greater. Preferably, the inhibitor reduces export of the reporter protein in a dose-dependent manner. Preferably, there should be no significant effect on the cytosolic specificity and toxicity control, e.g. luciferase. Candidate compounds, e.g. inhibitors, may be obtained as appropriate, e.g. from a variety of sources, including libraries of chemicals and natural extracts, low molecular weight compounds (LMW's), antibodies, recombinant DNA molecules and expression libraries, DNA, RNA, etc. In order to ascertain that the effect of a candidate compound which is found to inhibit the production of secreted and/or membrane protein according to the present invention is specific for the SP being used in the assay, further DNA which encodes a fusion protein containing, e.g. consisting, e.g. essentially, of, a signal peptide which is different to the signal peptide used for screening according to the present invention and which is linked to a reporter gene protein which is different from the reporter gene protein used for screening according to the present invention, may be present in a cell used for screening according to the present invention. If a candidate compound inhibits production of one of the reporter gene proteins and does not inhibit production of the other reporter gene protein present, there is evidence that the inhibitory effect is not due to a toxic effect of the candidate compound to the cell; otherwise, if the production of both reporter genes is inhibited, there is strong indication that the candidate compound has either a toxic effect on the cell used, or a non-specific inhibitory effect on the cotranslational translocation process.

In another aspect the present invention provides a process for the identification, e.g. and optionally selection, of compounds which interfere in the production of secreted or membrane protein, e.g. in cotranslational translocation; in cells comprising DNA which encodes a fusion protein containing, e.g. consisting, e.g. essentially, of, a signal peptide fused to a reporter gene protein, which cells are allowed to produce said reporter gene; which process comprising determining whether there is a difference in the amount of reporter gene protein produced with or without the presence of a candidate compound, respectively, e.g. and optionally selecting a compound in the presence of which the amount of reporter gene protein produced is different compared with the amount of reporter gene protein produced in the absence of said compound, e.g. and using such compound as a pharmaceutical, e.g. after chemical dervatisation.

In another aspect the present invention provides an assay for the identification of compounds which interfere with the process of cotranslational translocation and with the production of secreted or membrane protein which assay comprises as a substantial element cells containing DNA which encodes a fusion protein containing, e.g. consisting, e.g. essentially, of, a signal peptide fused and/or linked to a reporter gene protein; e.g. and, if desired, which further comprises means for cell treatment, e.g. including cell stimulation, to produce said reporter gene protein; e.g. and means for the detection of said reporter gene protein in an appropriate environment.

In another aspect the present invention provides an assay as described above, further comprising DNA encoding a second fusion protein containing, e.g. consisting, e.g. essentially, of, a signal peptide fused and/or linked to a reporter gene protein, wherein said signal peptide is different and the reporter gene is different from the signal peptide and from the reporter gene protein in the first fusion protein; e.g. and which further comprises means for cell treatment, e.g. including cell stimulation, if desired, to produce said reporter gene protein; e.g. and means for the detection of said reporter gene protein in an appropriate environment.

In another aspect the present invention provides an assay as described above, comprising a first fusion protein as described above and further comprising DNA which encodes a protein containing, e.g. consisting, e.g. essentially, of, a reporter gene protein which is different to a reporter gene protein in the first fusion protein, and whose expression is driven by a promoter which is the same or which has the same specific function (i.e. initiating transcription), e.g. the same eukaryotic promoter, either constitutive or inducible, as the expression of the first fusion protein; e.g. and further comprising a specifity/toxicity control protein whose expression is driven by a promoter as described above; e.g. and, if desired, which further comprises means for cell treatment, e.g. including cell stimulation, to produce said reporter gene proteins,e.g. and said specifity/toxicity control protein; e.g. and means for the detection of said reporter gene proteins, e.g. and said specifity/toxicity control protein; in an appropriate environment.

The DNA of said first and said second fusion protein, e.g. and of said specifity/toxicity control protein, may be located in the same (host) cell, or in different cells, i.e. a mix of different cells may be used.

An assay as defined above may be in the form of a kit, e.g. a screening kit.

In another aspect the present invention provides a kit, e.g. a screening kit, comprising an assay as defined above, which further comprises means for cell treatment, e.g. including cell stimulation, and/or culture to produce said reporter gene protein(s), e.g. and said specifity/toxicity control protein; and means for the detection of said reporter gene protein in an appropriate environment.

The processes, assays and kits according to the present invention are useful for the identification-of pharmaceutically active compounds (pharmaceuticals) by screening. Pharmaceutically active compounds include compounds active in all kinds of disease where the expression of the secreted and/or membrane protein(s) is relevant, e.g. diseases mediated via IL-4, IL12p40, MCP1, VCAM-1, VEGF, such as allergic and inflammatory diseases, atopic dermatitis, psoriasis, atherosclerosis, asthma, rheumatoid arthritis, multiple sclerosis, inflammatory bowel disease; and/or in cancers and in the prevention of tissue graft rejection.

In the following examples all temperatures are given in degree Centigrade and are uncorrected.

The following abbreviations are used:

| | |
|---|---|
| aa: amino acid | AmS: ammonium sulfate |
| CMV: cytomegalovirus | DTE: EDTA |
| Endo F: Endoglycosidase F | ER: endoplasmic reticulum |
| ICAM-1: Intracellular | IGEPAL: Octylphenylpolyethylene glycol |
| adhesion molecule 1 | |
| IL-4: Interleukin 4 | IL-12p40: Interleukin 12 p40 |
| MCP-1: monocyte chemo- | PAGE: polyacrylamlde gelelctrophoresis |
| attractant protein 1 | |
| PBS: phosphate buffered | PCR: polymerase chain reaction |
| saline | |
| PMSF: phenylmethyl- | pNPP: p-nitrophenyl-phosphate |
| sulfonylfluoride | |
| RT: reverse transcription | SDS: sodium dodecyl sulfate |
| SEAP: secreted alkaline | SR: signal recognition particle receptor |
| phosphatase | |
| SRP: signal recognition | SP: signal peptide |
| particle | |
| TCA: trichloro acetic acid | TEA: triethanolamine |
| TGF-α: transforming | TNF-α: tumor necrosis factor α |
| growth factor α | |
| VEGF: vascular endothelial | wt: wild type |
| growth factor | |
| VCAM-1: vascular cellular | |
| adhesion molecule-1 | |

A “candidate compound” may be a compound as disclosed in WO 96/03430, e.g. a compound of formula

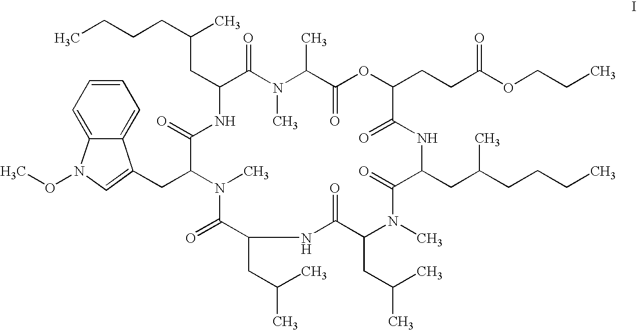

### EXAMPLE 1

Construction of Expression Plasmids and Fusion Genes

The E-Selectin and ICAM-1 expression vectors pCDM8-E-selectin and pCDM8-ICAM-1 allowing transient expression in mammalian cells and cell-free translation are obtained from R&D Systems. VCAM-1 cDNA is obtained by reverse transcription of total RNA from TNF-α stimulated primary human umbilical vein endothelial cells (HUVEC) as follows. After extraction of total RNA with Trizol (GibcoBRL) according to the suppliers recommendations, reverse transcription (RT) PCR on approximately 3 μg total HUVEC RNA is performed using the Advantage High Fidelity PCR kit (CLONTECH) under standard conditions with N- and C-terminal VCAM-1-specific sense and antisense oligonucleotide primers, respectively. Primers are tailed, introducing a Kpnl restriction site 5′ of the Kozak sequence and a Xhol site 3′ of the stop codon. Placental SEAP is amplified accordingly by PCR using plasmid pBC 12/PLAP489 (Berger et al., 1988) as a template and similar tailed SEAP-specific sense and antisense oligonuclotide primers. The SEAP and VCAM-1 cDNAs are isolated and cloned into the pCR2.1 vector (TA cloning kit, Invitrogen). All sequences are confirmed by sequencing and subsequently subcloned into the mammalian expression vector pcDNA3.1 (+) (Invitrogen) as Kpnl Xhol fragments for transient expression under control of the constitutive immediate-early CMV promoter and for cell-free translation from the bacteriophage T7 promoter. To generate SP-gene fusion constructs the recombinant PCR-ligation-PCR mutagenesis method is used (Ali and Steinkasserer, 1995). Briefly, in a primary PCR reaction the two fusion gene fragments are independently amplified using appropriate specific primers for the SPs (PCR product A) and the mature sequences (PCR product B), respectively. Approximately equal molar quantities of each PCR product A and B are phosphorylated and ligated. Finally, out of the possible ligation combinations, the desired fusion gene construct is specifically amplified from an aliquot of the ligation reaction by a secondary PCR using the 5′ sense primer of the SP and the 3′ antisense primer of the mature sequence. With these primers a Kpnl site 5′ of the Kozak sequence and a Xhol site 3′ of the stop codon are introduced as described above. The complete fusion gene PCR products are subcloned as Kpnl Xhol fragments into the expression vector pcDNA3.1 (+) and all sequences are confirmed by sequencing.

### EXAMPLE 2

Cell Culture and Transient Transfections HEK293 cells are maintained in Dulbeccos' modified Eagle's medium (DMEM, Gibco-BRL), supplemented with 10% heat-inactivated fetal calf serum (FCS, Bio-Whittaker) and 100 Units/ml each of penicillin (BC) and streptomycin (Gibco-BRL) at 37° in a 5% humidified CO₂ incubator. HUVEC cells are cultured in endothelial cell basal medium (EBM, Clonetics Corp.) supplemented with 10% FCS, 5×10⁻⁴ M dibutyryl cAMP (Sigma), 1 μg/ml hydrocortisone (Sigma) and 10 ng/ml human EGF (Boehringer) at 37° in a 5% humidified CO₂ incubator. VCAM-1 production is stimulated by incubation with 100U/ml TNF-α for ca. 8 hours. For transient transfection, HEK293 cells are seeded into 6-well or 24-well cell culture dishes at a density of 6×10⁵ cells/well or 1.5×10⁵ cells/well, respectively, the day prior to transfection and grown to 50-70% confluency. Cells are transfected with 2 μg or 1 μg of plasmid DNA, respectively, using the SuperFect reagent (Qiagen) according to the supplier's recommendations. Cells are incubated either without or with addition of a candidate compound, respectively, at concentrations indicated for ca. 24 to 48 hours. For proteasome inhibition experiments Lactacystin (Calbiochem) is added at a final concentration of 5 μM alone or together with a candidate compound 5 hours post-transfection, respectively.

### EXAMPLE 3

Protein Analysis

Protein expression in cells is analyzed by Western blot and subsequent immunoblot analysis. Cells are scraped off in PBS containing 0.25 M of NaCl, pelleted, resuspended in 50 μl of lysis buffer (100 mg deoxycholic acid/180 ml PBS, 5 M NaCl, 1% IGEPAL, 30 μl protease inhibitor cocktail, 1 tablet complete, mini, EDTA-free (Boehringer Mannheim)) in 500 μl of H₂O and incubated on ice for 30 minutes. After intensive vortexing of the samples and centrifugation for 6 min at 13000 rpm, 4° (Eppendorf centrifuge 5402), supernatants are transferred into new tubes and protein concentrations are determined (BCA Assay, Pierce).

Cell lysates are mixed 5:1 with reducing 5x Laemmli sample buffer (0.2 M Tris-HCl pH 8.8, 5 mM EDTA, 1 M Succrose, 1 mM DTE, Bromophenol blue+⅙ 20% SDS) or 1:2 with non-reducing 2X Laemmli Sample Buffer (BioRad), respectively, heated at 99° for 5 minutes and electrophoretically separated on SDS-PAGE (4-20% gradient Ready Gels; BioRad). Proteins are blotted on Protran nitrocellulose transfer membrane (Schleicher & Schuell) using a semi-dry transfer cell (Trans-Blot SD, BioRad; semi-dry blotting buffer 48 mM Tris, 39 mM glycine, 1.3 mM SDS, 20% Methanol, pH 9.2) or a tank blot transfer cell (Mini Trans-Blot Electrophoretic Transfer Cell, BioRad; tank blot buffer: 25 mM Tris, 200 mM Glycine, 20% Methanol). Blotting efficiency is controlled by protein staining with Ponceau S solution (Sigma). Expression of the respective recombinant protein in transiently transfected cells is determined by immunoblot analysis using the appropriate specific antibody followed by a horseradish peroxidase conjugated secondary antibody and the ECL Western blotting detection kit (Amersham Pharmacia Biotech) according to the manufacturer's instructions and subsequent fluorography. Protein expression is quantified by scanning densitometry. Glycosylation of protein is analyzed by deglycosylation with Endo F (Boehringer Mannheim). Equal amounts of cell lysate and 2x Endo F Buffer (100 mM KPO₄, 7.4, 20 mM EDTA, 0.4% SDS) are heated for 1 min at 100°. SDS is neutralized with 0.5-2% IGEPAL (‘Nonidet P 40’ or Octylphenylpolyethylene glycol, Sigma) and half of the sample is incubated with 1 U Endo F per 7 μl sample at 37° for 1 hour. The untreated half of the sample serves as negative control. Samples are separated by SDS-PAGE and proteins are detected by Western blot analysis as described above.

### EXAMPLE 4

Detection of Secreted Reporter Protein SEAP

Human placental SEAP levels in cell supernatants are determined using either a fluorescence-based assay or an assay that measures light absorbance at 405 nm accompanying hydrolysis of pNPP according to the method described (Berger et al., 1988). Briefly, a 500 μl aliquot of medium is removed from the culture dish, clarified for 1 min at 14,000×g and heated for 5 minutes at 65°. An aliquot of medium (10 μl-100 μl) is adjusted to 1x SEAP buffer (1.0 M diethanolamine, pH 9.8, 0.5 mM MgCl₂, 10 mM L-homoarginine) in a final volume of 200 μl in a 96-well flat bottom culture dish (Nunc) and prewarmed to 37° for 10 min. 20 μl of 120 mM pNPP in 1x SEAP buffer prewarmed to 37° is added and the change in absorbance at 37° is plotted. Heating step and inclusion of L-homoarginine in the assay buffer inhibit any endogenous phophatase activity. For determination of SEAP levels by a fluorescence-based assay the Atto Phos AP Fluorescent Substrate Sysem (Promega) is used according to the manufacturer's instructions.

### EXAMPLE 5

Cell-Free Translation/Translocation Assay

Cell-free translations are carried out in the coupled TNT reticulocyte lysate system (Promega) using bacteriophage T7 polymerase according to the suppliers recommendations in a final volume of 25 μl and in the presence of [³⁵S]-methionine (Redivue, 10 mCi/ml, Amersham). To study cotranslational translocation across the ER membrane dog pancreatic microsomes (Promega) are present during translation reactions at concentrations between 1 to 2.5 μl per 25 μl reaction mixture. After translation/translocation, 2.5 μl of the reaction mixture are denatured in SDS loading buffer (12.5 mM Tris-HCl, pH 6.8, 80 μM EDTA, 26 mM DTT, 1% SDS, 100 g/ml bromphenol blue, 0.01% NaN₃) for 5 minutes at 95° and subjected to SDS-PAGE (4-20% gradient or 15% Excel gels, Pharmacia). Gels are analyzed by autoradiography and quantitated using an Instant Phosphoimager (Packard). For sedimentation experiments 10 μl-15 μl of the reaction mixture are diluted with 100 μl of 0.25 M sucrose, 20 mM EDTA, 50 mM triethanolamine (TEA), pH 7.5, incubated for 10 minutes on ice and overlaid onto a 100 μl sucrose cushion (0.5 M sucrose, 140 mM sodium acetate, 20 mM EDTA, 2.5 mM MgOAc, 50 mM TEA) in a Beckman TL100 polyallomer tube. Samples are centrifuged for 5 min at 100,000 rpm at 4° (Beckman TL100). The supernatant including the cushion is precipitated by addition of two volumes of saturated AmS solution on ice for at least 30 minutes. The precipitate obtained is collected by centrifugation at 4° for 15 minutes at 10,000 rpm and washed alternately with 1 ml of 5% ice-cold TCA and 1 ml of acetone. After air-drying the precipitate is dissolved in SDS sample buffer enriched with Tris-HCl, pH 7.5 and subjected to SDS-PAGE and autoradlography as described above. For protease protection assays, translation translocation reactions are placed on ice and supplemented with CaCl₂ to a final concentration of 2 mM. Proteinase K (Boehringer Mannheim) is added to a final concentration of 12.5 μg/ml and digestions are performed for 30 minutes on ice. Proteolysis is terminated by incubation with PMSF at a final concentration of 10 mM for 10 minutes on ice, subsequent addition of 30 μl SDS sample buffer and immediate heating to 95° for 5 minutes. Samples are subjected to SDS-PAGE and autoradiography as described above.

### EXAMPLE 6

Inhibition of VCAM-1 Glycoprotein Production

A novel substance class of fungus, derived cyclopdepsipeptides has been described recently, which potently and preferentially inhibit expression of the adhesion molecule VCAM-1 on human endothelial cells relative to ICAM-1 and E-Selectin (Boger et al., 1999; Foster et al., 1994).

Subsequently, it was shown that derivatives of this compound class (“candidate compound”) primarily suppress VCAM-1 production at a post-transcriptional level. In transient expression experiments using HEK293 cells and plasmids expressing both VCAM-1 and E-Selectin under control of the heterologous CMV promoter (see FIG. 1) the inhibitory effects of the compound class can be reproduced supporting the notion that the compound class primarily acts prost-transcriptional. HEK293 cells are transfected as described above with plasmids expressing either VCAM-1 or E-Selectin. A candidate compound is added at increasing concentration as indicated in FIG. 1. After 48 hours post-transfection expression of VCAM-1 and E-Selectin is monitored by Western blot and subsequent immunoblot analysis and quantified by extrapolation against expression of the house keeping gene β-actin. As shown in FIG. 1, in transfected HEK293 cells VCAM-1 and E-Selectin proteins are synthesized as fully glycosylated 100 and 115 kDA proteins, respectively (see FIG. 1, lane 2, arrows). Whereas expression of VCAM-1 is inhibited by increasing concentrations of the candidate compound-CP, synthesis of E-Selectin is not-affected (see FIG. 1, lanes 3-6). The candiate compound inhibits 50% of VCAM-1 glycoprotein synthesis in the low nanomolar range of <5 nM comparable to results obtained in endothelial cells (Foster et al., 1994).

### EXAMPLE 7

Inhibition of Cotranslational Translocation of VCAM-1

Type I transmembrane proteins such as the adhesion molecules VCAM-1, ICAM-1 and E-Selectin are translated and inserted into the cell membrane cotranslationally at the level of the ER membrane. To investigate the possibility that the candidate compound is interfering at this early stage of protein expression the effect of the drug on the process of cotranslational translocation in a cell-free assay system is analysed. Full length cDNAs of the adhesion molecules VCAM-1, ICAM-1 and E-Selectin are transcribed and translated in a cell-free reticulocyte lysate system in the absence or presence of dog pancreatic microsomes, respectively (Promega). Proteins are radioactively labelled and visualised by autoradlography. As shown in FIG. 2A (compare lanes 1 and 2) in the presence of microsomes, but not in their absence, two protein bands for VCAM-1, ICAM-1 and E-Selectin are detected. The shift in molecular weight is due to occurring core-glycosylation in the lumen of the microsomal membranes. Since glycosylating enzymes are active only within microsomal membranes in the cell-free system, these results suggest that all three full-length proteins are effectively targeted, translocated and subsequently core-glycosylated in the lumen of the ER-derived microsomal vesicles. To test efficient translocation protease is added after translation to assay for translocated protein that is protected from degradation by the phospholipid bilayer of the microsomal vesicles (Nicchitta and Blobel, 1990). As shown in FIG. 2B (compare lanes 1 and 2) the lower molecular weight, unglycosylated and not translocated form of the respective adhesion molecule is fully digested, in contrast the higher molecular weight protein moiety and core-glycosylated form is not susceptible to proteolytic degradation. Furthermore, the core-glycosylated form (in contrast to the unglycosylated form) of each protein sediments with the membrane fraction in sucrose gradient centrifugation experiments (see FIG. 2C).

Altogether these results show that VCAM-1, ICAM-1 and E-Selectin proteins are effectively cotranslationally translocated into the lumen of the ER-derived microsomal vesicles in the cell-free system as demonstrated by the appearance of the core-glycosylated, protease-protected and membrane-associated forms. To study the effect of a candidate compound on cell-free cotranslational translocation of the proteins a candidate compound (CP) is added to the translation/translocation assay. As shown in FIGS. 2A and 2B only the core-glycosylated and protease-protected form of VCAM-1 is inhibited by increasing concentrations of the candidate compound indicating that translocation across the microsomal membranes is inhibited but translation itself is not. In contrast, translocation of ICAM-1 is only inhibited at 20 to 30 times higher concentrations of the candidate compound used and E-Selectin is shown to be resistant at the concentrations tested (see FIG. 2A, B and C).

These data obtained with the cell-free translation/translocation assay strongly suggest that the candidate compound specifically interferes with cotranslational translocation across the ER membrane in cells resulting in mislocalization of non-glycosylated, full-length precursor protein to the cytosolic compartment where it is expected to be incorrectly folded and therefore expectedly is rapidly degraded. This finding might also explain why no VCAM-1 protein is detectable in cells treated with sufficient amount of a candidate compound (see FIG. 1, lane 5). Most misfolded proteins are degraded in the cytosol of the cell by the ubiquitin-proteasome pathway (Voges et al., 1999). To provide direct evidence that the candidate compound class selectively interferes with cotranslational translocation of VCAM-1 in cells resulting in mislocalization, misfolding and degradation of the protein, cells transiently transfected with VCAM-1 cDNA are co-treated with a candidate compound and lactacystin, a specific inhibitor of the proteasome degradation pathway (Lee and Goldberg, 1998). As shown in FIG. 3 (upper panel, compare lanes 3 and 5) co-treatment of these cells with lactacystin results in the accumulation of a polypeptide moiety of lower molecular weight that is not present after treatment with the candidate compound alone. By deglycosylation (see FIG. 3, lower panel) experiments with Endo F this polypeptide was subsequently identified as the full length, non-glycosylated VCAM-1 protein. These data provide first direct evidence that the candidate compound class inhibits VCAM-1 glycoprotein synthesis in cells by specifically interfering with cotranslational translocation.

### EXAMPLE 8

Identification of the Cyclopeptolide Sensitive Domain of VCAM-1

Leader sequences or SP's play a central role in the targeting and translocation of soluble and integral membrane proteins exported from the cell by the classical ER/Golgi pathway. To demonstrate a potential role of the SP in conferring drug sensitivity chimeric fusion constructs are designed combining SP's and mature sequences of the compound-sensitive VCAM-1 and compound-resistant E-Selectin cDNAs (see FIG. 4). The effects of the candidate compound on these chimeric fusion constructs is tested in both the cell-free translation/translocation assay and in transiently transfected HEK293 cells (see FIG. 4). The VCAM-1 mutant molecule containing the SP from E-Selectin is shown to be resistant to a candidate compound, e.g. a compound of formula I, whereas the E-Selectin mutant molecule with SP from VCAM-1 is found to be rendered partially sensitive to said compound. These results suggest that the signal peptide is required for conferring drug sensitivity but is not sufficient and adjacent sequences from the mature domain of VCAM-1 might be required for full candidate compound sensitivity. Subsequently, the minimal candidate compound-sensitive domain of VCAM-1 showing full sensitivity to candidate compound-treatment has been defined as 15 aa of the SP plus 4 aa of the adjacent mature domain of VCAM-1 (see FIG. 4, VCAM_{SP15+4}/E-Sel).

### EXAMPLE 9

Establishment and Validation of SP-SEAP Assay

For the establishment and as a first proof of principle of an assay (system) according to the present invention, SP-reporter gene fusion constructs are generated using the SP of compound sensitive and insensitive proteins, respectively, and the mature sequence of placental SEAP as the reporter gene (see FIG. 5). It was found that these SP-SEAP fusion constructs are transiently overexpressed in cells in the absence or in the presence of increasing concentrations of a candidate compound. Finally, secretion of the reporter SEAP protein into the medium is determined. As shown in FIG. 6 the secretion of the reporter protein SEAP mirrors the compound sensitivity of the proteins from which the signal peptides are originally derived. As expected SEAP and the E-Selectin_{SP}-SEAP fusion proteins are found to be compound-insensitive, in contrast the VCAM_{SP}-shows slight sensitivity and the VCAM_{SP(15+4)}-SEAP construct full sensitivity to the candidate compound in a dose-dependent manner.

### EXAMPLE 10

Functionality of SP-SEAP Gene Fusion Constructs of Selected Protein Targets Playing Critical Roles in Various Disease Processes

To further validate the novel assay system claimed in the present invention we generated SP-SEAP fusion constructs similarily to the method as described in example 1, but using the SP of other protein targets playing critical roles in various disease processes and tested their functionality by transient overexpression in mammalian cells in the absence or presence of increasing concentrations of compound CP as described (FIG. 7). IL-4 was chosen as a target in allergic inflammatory diseases (e.g. allergy, asthma, multiple sclerosis, atopic dermatitis), IL-12p40 in-inflammation and-autoimmune diseases, MCP-1 in inflammatory diseases (e.g. atherosclerosis, rheumatoid arthritis, multiple sclerosis) and VEGF in conditions associated with pathological angiogenesis (e.g. metastasis of solid tumors, psoriasis). As shown in FIG. 7 all SP-SEAP gene fusion constructs were functional. Furthermore, whereas the IL4, IL-12p40 and MCP-1 SP-SEAP fusion constructs were insensitive to increasing concentration of compound CP the VEGF SP-SEAP constructs was sensitive to increasing concentrations of compound CP in a dose dependent manner as compared to the VCAM-1 SP-SEAP construct.

### BRIEF DESCRIPTION OF THE FIGURES (FIG.)

FIG. 1 shows Western blot analysis of HEK293 cells not transfected or transiently transfected with plasmids expressing either VCAM-1 or E-Selectin cDNAs following treatment with increasing concentrations of compound CP as indicated. Antibodies used were specific for either VCAM-1, E-Selectin or the endogenous β-actin. Molecular weight markers are indicated on the right in kDa.

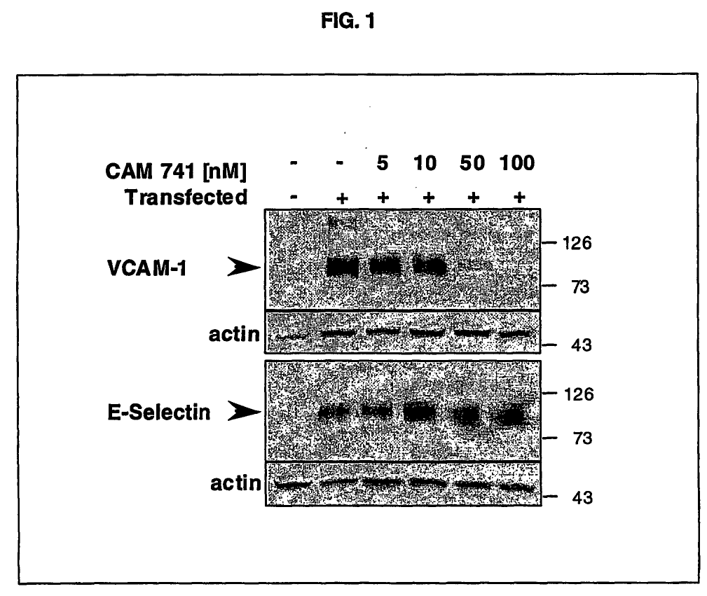

FIG. 2 shows an autoradiogram of a translation/translocation experiment. Radioactively labelled VCAM-1, ICAM-1 and E-selectin were synthesized using reticulocyte lysates in the absence or presence of microsomal membranes. Compound CP was added at increasing concentrations as indicated. Translation/translocation products were analyzed by SDS-PAGE followed by autoradiography either directly (A), after protease treatment (B) or after sedimentation centrifugation (C). S, supernatant; P, microsomal pellet. Arrows indicate core-glycosylated protein; asterisks depict unglycosylated protein. Molecular weight markers are indicated on the right in kDa.

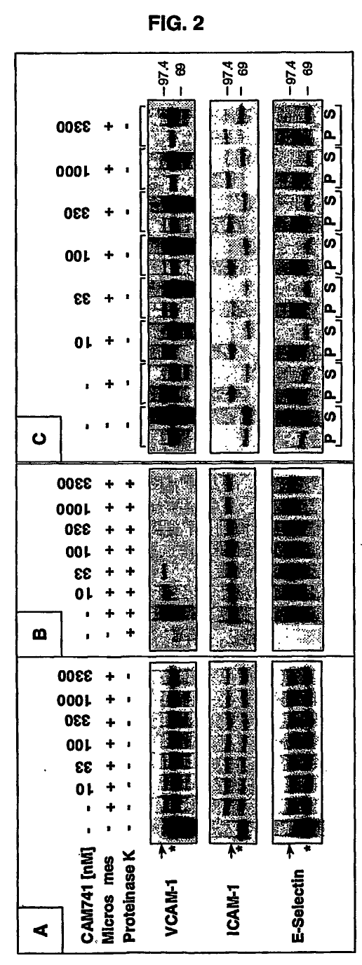

FIG. 3 shows in the upper panel a Western blot analysis of cells transiently transfected with a plasmid expressing VCAM-1 protein not treated or treated with either compound CP and lactacystin alone or co-treated with compound CP and lactacystin. The lower panel shows a Western blot analysis of aliquots of the translation/translocation samples after digestion with endoglycosidase F (Endo F).

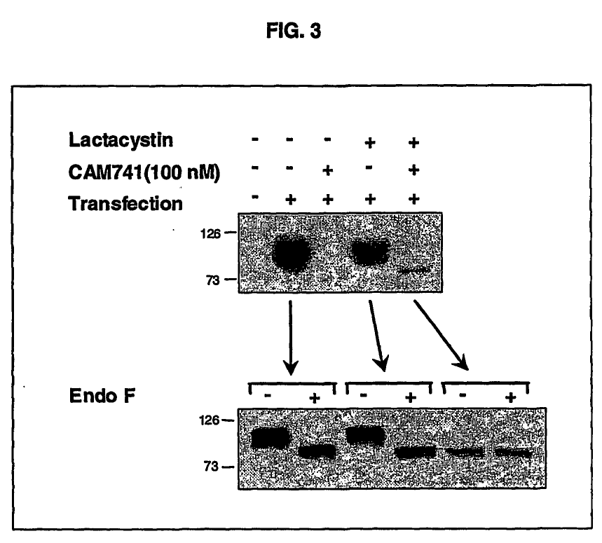

FIG. 4 shows schematically the SP-fusion construct used to define the compound sensitive domain of VCAM-1. On the right side there are indicated the concentrations of the compound CP inhibiting 50% of either the production of the mature fully glycosylated protein after transient expression of the fusion proteins in HEK293 cells or the core-glycosylated form after cotranslational translocation in the cell-free translation/translocation assay (n.d., not determined).

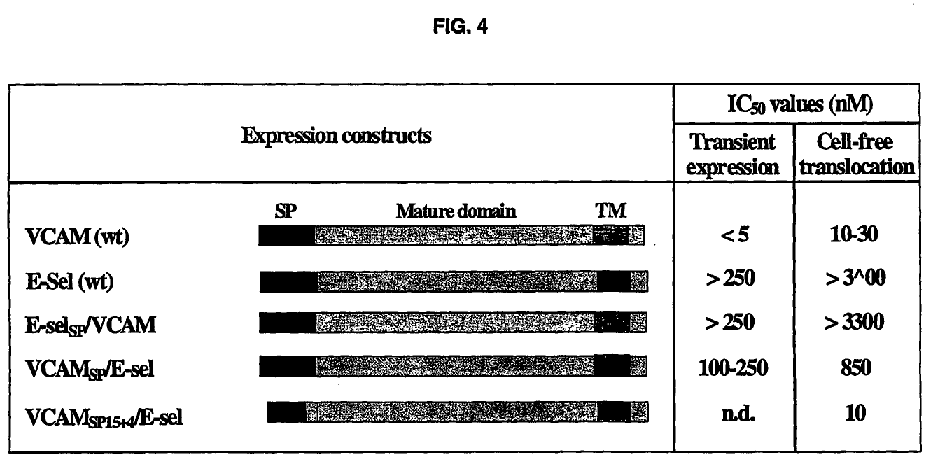

FIG. 5 shows schematically the SP-SEAP fusion constructs used to establish and validate assay systems according to the present invention.

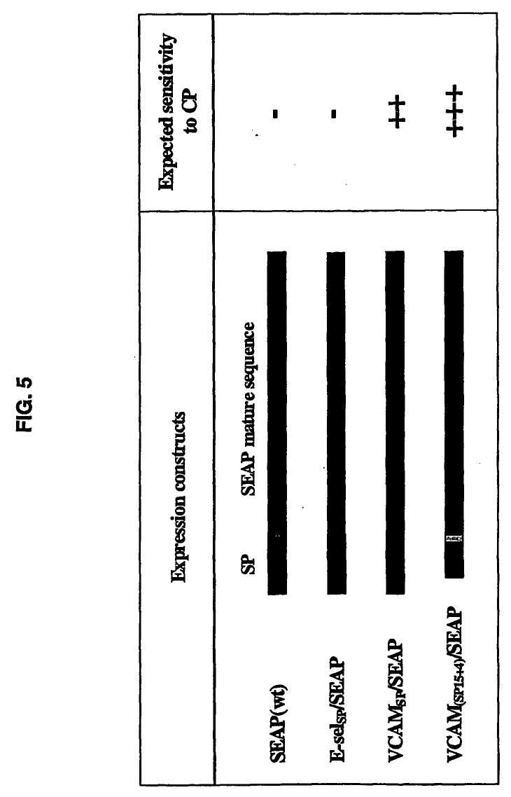

FIG. 6 is a graph showing SEAP activity in the supernatant of cells transiently transfected with plasmids expressing the SP-SEAP fusion constructs indicated following treatment with increasing concentrations of compound CP.

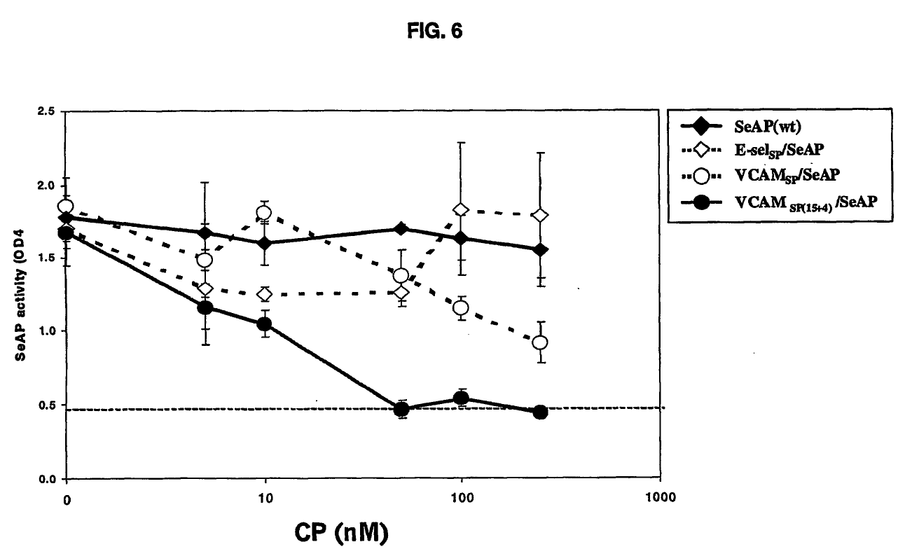

FIG. 7 is a graph showing SEAP activity in the supernatant of cells transiently transfected with plasmids expressing the SP-SEAP fusion constructs indicated following treatment with increasing concentrations of compound CP. SEAP activity is plotted as % of the control (ctl) sample without addition of compound CP. Results shown are means of triplicate wells. Error bars indicate SD.

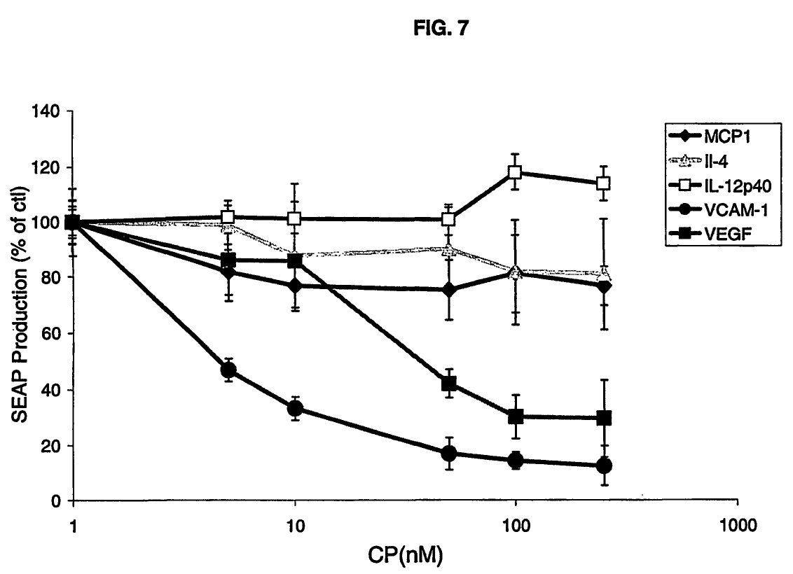

### REFERENCE LIST

Ali, S. A. and Steinkasserer, A. (1995). PCR-ligation-PCR mutagenesis: a protocol for creating gene fusions and mutations. Biotechniques 18, 746-750.

Berger, J., Hauber, J., Hauber, R., Geiger, R., and Cullen, B. R. (1988). Secreted placental alkaline phosphatase: a powerful new quantitative indicator of gene expression in eukaryotic cells. Gene 66, 1-10.

Boger, D. L., Keim, H., Oberhauser, B., Schreiner, E. P., and Foster, C. A. (1999). Total Synthesis of HUN-7293. Journal of the American Chemical Society 121, 6197-6205.

Foster, S. A., Dreyfuss, M., Mandak, B., Meingassner, J. G., Naegeli, H. U., Nussbaumer, A., Oberer, Scheel, G., and Swoboda, E. -M. (1994). Pharmacological modulation of endothelial cell-associated adhesion molecule expression; implications for future treatment of dermatological diseases. Journal of Dermatology 21, 847.

Lee, D. H. and Goldberg, A. L. (1998). Proteasome inhibitors: valuable new tools for cell biologists. Trends. Cell Biol. 8, 397-403.

Matlack, K. E., Mothes, W., and Rapoport, T. A. (1998). Protein translocation: tunnel vision. Cell 92, 381-390.

Nicchitta, C. V. and Blobel, G. (1990). Assembly of translocation-competent proteoliposomes from detergent-solubilized rough microsomes. Cell 60, 259-269.

Voges, D., Zwickl, P., and Baumeister, W. (1999). The 26S proteasome: a molecular machine designed for controlled proteolysis. Annu. Rev. Biochem. 68:1015-68., 1015-1068.

1. Use of a DNA construct encoding a reporter protein fused to a selected heterologous signal peptide in a screening process or screening assay for the screening of candidate compounds, to obtain compounds which interfere with the process of cotranslational translocation and with the production of secreted and/or membrane protein.

2. Use according to claim 1, wherein the compounds which interfere with the process of cotranslational translocation and with the production of secreted and/or membrane protein are pharmaceutically useful compounds.

3. A process for the identification of a compound which interferes with the production of secreted and/or membrane protein comprising determining the amount of protein secreted and the extent or degree of cotranslational translocation across the membrane of the endoplasmatic reticulum of said secreted and/or membrane protein in the presence and in the absence of a candidate compound.

4. A process for the identification and optionally for the selection of a compound which interferes with the process of cotranslational translocation and with the production of secreted and/or membrane protein by a cell comprising the steps of
a1. allowing or stimulating expression of the reporter gene protein in cells transfected with DNA which encodes a fusion protein containing a heterologous signal peptide linked to a reporter protein,
b1. detecting secreted reporter protein produced in step a1. in the absence or in the presence of a candidate compound, respectively, and determining whether there is a difference in the amount of reporter gene protein produced in step a1. in the absence or in the presence of a candidate compound, respectively, and optionally
c1. selecting a candidate compound, in the presence of which the amount of reporter gene protein produced in step a1. is different from the amount of reporter gene protein produced in step a1. in the absence of said candidate compound, and optionally using such selected compound as a pharmaceutical, optionally after chemical derivatisation.

5. A process for the identification of a compound which interferes with the process of cotranslational translocation and with the production of secreted and/or membrane protein by a cell, comprising the steps of
a. providing DNA which encodes a fusion protein containing a heterologous signal peptide linked to a reporter protein,
b. introducing DNA obtained in step a. into a DNA vector,
c. transfecting DNA obtained in step a. or in step b. into a cell;
d. allowing or stimulating expression of the reporter gene protein in a transfected cell obtained in step c. under appropriate conditions;
e. detecting secreted reporter protein produced in step d. in the absence or in the presence of a candidate compound, respectively; and
f. determining whether there is a difference in the amount of reporter gene protein produced in step e. in the absence or in the presence of a candidate compound, respectively, which amount is determined according to step e., and optionally
g; selecting a candidate compound, in the presence of which the amount of reporter gene protein produced in step d. is different from the amount of reporter gene protein produced in step d. in the absence of said candidate compound, and optionally using such selected compound as a pharmaceutical, optionally after chemical derivatisation.

6. A process according to any one of claims 4 to 5, wherein the DNA in step a., or step a1., respectively, comprises additional intervening DNA sequences which encode additional amino acids.

7. A process according to claim 6, wherein additional intervening DNA sequences which encode additional amino acids are obtained from the DNA sequence of the mature DNA sequence adjacent to that signal peptide of the secreted and/or membrane protein which is part of the fusion protein used in a process according to any one of claims 4 or 5 according to step a. or step a1., respectively.

8. An assay for the identification of compounds which interfere with the process of cotranslational translocation and with-the production of secreted or membrane protein, which assay comprises as a substantial element cells containing DNA which encodes a fusion protein containing a signal peptide fused and/or linked to a reporter gene protein.

9. An assay according to claim 8, further comprising DNA encoding a second fusion protein containing a signal peptide fused and/or linked to a reporter gene protein, wherein the signal peptide is different and the reporter gene protein is different from the signal peptide or from the reporter gene protein, respectively, in the first fusion protein.

10. A kit comprising an assay as defined in any one of claims 8 or 9, and further comprising
means for cell treatment, and/or culture to produce said reporter gene protein(s); and
means for the detection of said reporter gene protein in an appropriate environment.
